(19) United States
(12) Patent Application Publication
(10) Pub. No.: US 2025/0248984 A1
(43) Pub. Date: Aug. 7, 2025

(54) COMBINATION THERAPY FOR THE TREATMENT OF CANCER

(71) Applicants: Children's Hospital Medical Center, Cincinnati, OH (US); University of Cincinnati, Cincinnati, OH (US)

(72) Inventors: Nicolas Nassar, Cincinnati, OH (US); Jose A. Cancelas, Montgomery, OH (US); Shailaja Hegde, Alexandria, KY (US)

(21) Appl. No.: 19/046,763

(22) Filed: Feb. 6, 2025

Related U.S. Application Data
(60) Provisional application No. 63/550,925, filed on Feb. 7, 2024.

(51) Int. Cl. A61K 31/444; A61K 31/138; A61K 31/4535; A61K 31/565; A61P 35/02

(52) U.S. Cl. CPC A61K 31/444; A61K 31/138; A61K 31/4535; A61K 31/565; A61P 35/02

(57) ABSTRACT

The present technology is directed to a method of treating a cancer, or multiple cancers, comprising administering to said individual one or more compounds selected from a VAV3-inhibitor such as IODVA1, followed by administration of a selective estrogen receptor modulator (SERM) and/or selective estrogen receptor degrader (SERD), to an individual in need thereof. In embodiments, the administration may be concurrently with or following IODVA1 administration.

### CROSS-REFERENCE TO RELATED APPLICATIONS

The present patent application claims priority from U.S. Provisional Application No. 63/550,925, filed Feb. 7, 2024, which are incorporated herein by reference in their entirety.

### STATEMENT REGARDING FEDERALLY-SPONSORED RESEARCH

This invention was made with government support under CA237016 awarded by the National Institutes of Health. The government has certain rights in the invention.

### BACKGROUND

B-cell Acute Lymphoblastic Leukemia (B-ALL) presents significant challenges in treatment, particularly due to its propensity for relapse and dissemination to extramedullary organs like the central nervous system (CNS). While advancements have been made in therapies, the high relapse rates and the lack of therapies that eliminate leukemic stem/progenitor cells or treat relapsed and disseminated leukemia underscore the need for novel targeted treatment strategies.

### BRIEF SUMMARY

Leukemic cells from PDX mouse models of B-ALL treated with an inhibitor of the RAC activator VAV3 rely among others on the activity of the estrogen receptor a (ERa) for proliferation and relapse. Concurrent or sequential inhibition of VAV3 and ERa prolongs the survival of several PDX models of relapsed/refractory B-ALL long after treatment stoppage. Furthermore, the activity of ERa is found increased in a BCR-ABL1-driven leukemic model of Vav3-null leukemia and fulvestrant treatment prolongs the survival of this model. VAV3 inhibition increases the activity of ERa and concomitant synthesis of the sex hormone 17ß-estradiol/E2 by lymphoblastic cells. Our results highlight novel RAC-independent activities of a RacGEF and open the door for a novel treatment for B-ALL based on VAV3 and ERa concomitant inhibition.

The present technology is directed to a method of treating a cancer, or multiple cancers, comprising administering to said individual one or more compounds selected from

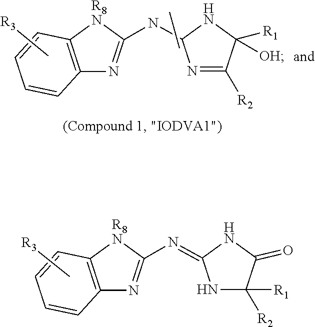

wherein R₁ and R₂ are independently substituted or unsubstituted aryl or heteroaryl rings;
wherein R₃ is singly or multiply substituted as H, D, Halo, CN, C1-C4 Alkyl, C1-C4 alkoxy, C1-C4 alkylsulfonyl, C1-C4 Alkyl amino, or C1-C4 mercapto; and wherein R₈=H, Me; and all tautomers thereof; followed by administration of a selective estrogen receptor modulator (SERM) and/or selective estrogen receptor degrader (SERD), to an individual in need thereof.

More particularly, the compound may be

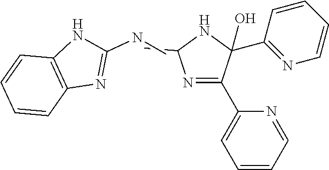

or a tautomer thereof; or

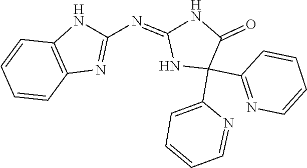 [SMILES: O=C1N/C(=N/c2nc3ccccc3[nH]2)NC1(c1ccccn1)c1ccccn1]

or a tautomer thereof.

The cancers treated using this method cancer may be selected from an H/K/NRAS-driven cancer, B-cell acute lymphoblastic leukemia (B-ALL), de novo B-ALL and therapy resistant B-ALL, TKI-resistant B-ALL, leukemia, lung cancer, non-small cell lung cancer (NSCLC), solid tumor, breast cancer, triple-negative breast cancer (TNBC), hormone-resistant triple negative breast cancer, and combinations thereof. The solid tumor may be selected from lung cancer, breast cancer, subtypes thereof, and combinations thereof. The cancers treated by this method may be characterized by expression of guanine nucleotide exchange factor VAV3 (VAV3), of its activated phosphorylated form(s), and estrogen receptor alpha (ERa).

Generally, the method of treating involves concurrent or sequential administration of IODVA1 and the SERM and/or SERD, where sequential treatment requires that the SERM and/or SERD be administered following administration of IODVA1. The said SERM and/or SERD may be administered at least one week following administration of IODVA1, or at least two weeks following administration of IODVA1, at least three weeks following administration of IODVA1, or at least four weeks following administration of IODVA1. The SERD may be selected from one or both of fulvestrant, elacestrant, giredestrant, imlunestrant, camizestrant, and clomiphene. The SERM may be selected from tamoxifen, raloxifene and toremifene, and combinations thereof.

In other embodiments, the SERM and/or SERD may be administered for at least two weeks, at least three weeks, at least four weeks, at least five weeks, or at least six weeks, where the administration may be either concurrently with and/or following IODVA1 administration. The administration may be selected from oral, intravenous, subcutaneous, intramuscular, pulmonary (inhalation), topical, transdermal, intranasal, ophthalmic, buccal, epidural, intrathecal, and combinations thereof. The IODVA1, SERM, and/or SERD may each being administered at a dose of from about 1 mg/kg to about 200 mg/kg, or from about 3 mg/kg to about 150 mg/kg, or from about 5 mg/kg to about 100 mg/kg, or from about 10 mg/kg to about 75 mg/kg, or from about 15 mg/kg to about 50 mg/kg.

In another embodiment, the present technology comprises a pharmaceutical combination comprising a compound having the formula

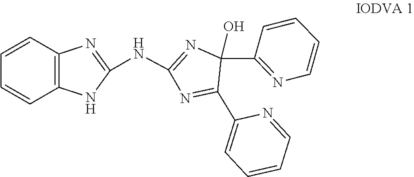 [SMILES: OC1(c2ccccn2)N=C(Nc2nc3ccccc3[nH]2)N=C1c1ccccn1]

and a selective estrogen receptor modulator (SERM) and/or selective estrogen receptor degrader (SERD). The IODVA1 compound and the SERM and/or SERD may be provided in separate dosage forms.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts the transcriptomic approach used to determine the mechanisms of resistance in leukemic cells from vehicle treated mice, mice after one week of IODVA1 treatment, mice after one week of ponatinib treatment, mice after one week of IODVA1/ponatinib combination treatment, mice in relapse after IODVA1 treatment, mice in relapse after ponatinib treatment, and mice in relapse after the IODVA1/ponatinib combination treatment.

FIG. 2 is a heatmap representation of the transcriptomics analysis used to identify genes driving resistance to IODVA1.

FIG. 3 is a heatmap representation of the differential transcriptional signature of the procollagen-proline 4-dioxygenase complex pathway between leukemic cells from mice treated with IODVA1 for one week (IW1, IW2, and IW3) and cells from mice that relapsed following IODVA1 treatment (IR1, IR2, and IR3). Leukemic cells from mice treated with vehicle control (V1, V2, and V3) are shown as reference.

FIGS. 4A-4C show the IODVA1/fulvestrant sequential treatment increases the survival of PDX models of TKI-resistant Ph+B-ALL. FIG. 4A depicts a Kaplan-Meier survival plot of PDX 2017-192-engrafted mice during treatment and after treatment withdrawal. NSG mice (N=6 per group) engrafted with B-ALL patient sample 2017-129 were treated days 20-58 (T1) with vehicle (black line), fulvestrant (blue line), the fulvestant/ponatinib combination (red line), IODVA1 (lavender line), the ponatinib/IODVA1 combination (green line), and the IODVA1/fulvestrant concurrent combination (teal line). For the IODVA1/fulvestrant sequential group, IODVA1 was treated first days 20-58 (T1) followed by fulvestrant days 58-105 (T2). FIG. 4B depicts leukemic burden assessment in the peripheral blood of PDX-implanted mice at the indicated time points. FIG. 4C shows leukemic burden assessment in the brain of PDX-implanted mice treated with vehicle control or the IODVA1/fulvestrant sequential treatment at the time of death.

FIG. 5 depicts the methods used to generate PDX models of B-ALL and test the fulvestrant/IODVA1 combination. Irradiated NSG mice were engrafted with B-ALL patient samples. When leukemic burden in peripheral blood reached 10-to-15% mice (3 males and 3 females) were divided into treatment groups and given fulvestrant (days 20-51), the IODVA1/fulvestrant concurrent combination, and the IODVA1/fulvestrant sequential combination.

FIG. 6 shows the Kaplan-Meier survival curve (left panel) and the leukemic burden (right panel) of Ph+B-ALL PDX 2018-136 engrafted NSG mice and treated with vehicle (black line), fulvestrant (red line), the IODVA1/fulvestrant concurrent combination (green line), and the IODVA1/fulvestrant sequential combination (lilac line).

FIG. 7 shows the Kaplan-Meier survival curve (left panel) and the leukemic burden (right panel) of Ph-like B-ALL PDX 2016-79 engrafted NSG mice and treated with vehicle (black line), fulvestrant (red line), the IODVA1/fulvestrant concurrent combination (green line), and the IODVA1/fulvestrant sequential combination (lilac line).

FIG. 8 shows the Kaplan-Meier survival curves of oncogenic KRAS-driven B-ALL PDX 2014-11 (left panel) and PDX 2019-163 (right panel) engrafted NSG mice and treated with vehicle (black line), IODVA1 (lilac circles), and the IODVA1/fulvestrant sequential combination. IODVA1 and fulvestrant were administered days 28-56 (T1) and 56-147 (T2), respectively.

FIG. 9 shows the data illustrating that B-ALL cells treated with IODVA1 and relapsed become addicted to ERa signaling. A shows proliferation of PDX cells purified from the bone marrow of PDX 2017-129 mice treated with vehicle or IODVA1 at relapse and treated with IODVA1 or fulvestrant as indicated. B shows that levels of apoptosis were quantified in the same cells as indicated.

FIG. 10 shows the data illustrating that VAV3 inhibition by IODVA1 increases the activity of ERa. Relative change in transcript levels of indicated genes in human leukemic cells purified from the bone marrow of PDX 2017-129 engrafted mice treated with vehicle or with IODVA1 after one week or at relapse.

FIG. 11 depicts the luciferase assay protocol and results obtained, demonstrating that IODVA1 stimulates ERa transcriptional activity and protein levels in leukemic NALM6 cells.

FIG. 12 shows the data illustrating that VAV3-inhibition increases levels of the sex hormone estradiol/E2 in the plasma of PDX 2017-129 engrafted mice treated as indicated.

FIG. 13 shows the data illustrating that VAV3 inhibition increases levels of secreted estradiol/E2. Levels of estradiol/E2 were measured by an ELISA assay in the media of NALM6 cells transfected with scramble shRNA or shVAV3 and treated as indicated.

FIG. 14 shows the data illustrating that IODVA1 treatment increases the transcripts of enzymes responsible for estradiol/E2 synthesis. Relative fold change in the transcript levels of ESR1, HSD17B1, and CYP19A1 in the human leukemic cells purified from the bone marrow of PDX 2017-129 engrafted mice and treated with IODVA1 for one week or at relapse.

FIG. 15 shows the data illustrating that ERx and VAV3 interact and that IODVA1 inhibits the translocation of the complex to the nucleus. A-NALM6 cells were treated with vehicle control, estradiol/E2 (1 nM, 1 hour), IODVA1 (1 uM, 1 h), or IODVA1 followed by E2 and the complex formation was detected by proximity ligation assay (PLA). B-NALM6 cells were lysed and co-immunoprecipitated with anti-VAV3 and anti-ERa antibodies and blotted with the indicated antibodies.

FIG. 16 shows the data illustrating that VAV3 and ERa interact in ER+but not in ER-cells.

### DETAILED DESCRIPTION

Disclosed are novel combination treatments for cancer. In embodiments, the novel combination treatments are useful for treatment of cancer resistant to currently available treatments. In embodiments, the cancer is leukemia.

IODVA1 is a small molecule inhibitor of the VAV3/RAC signaling pathway developed by Applicant, described, in Gasilina et al., PLOS One, 2020, PMID 32163428 and U.S. Patentapplicationn Pub. No. 2020/0345712 A1. IODVA1 significantly prolongs the survival of mouse models of B-cell acute lymphoblastic leukemia (B-ALL) including patient-derived xenograft (PDX) models of resistance to tyrosine kinase inhibitors (TKIs) and synergizes with Abl-TKIs such as dasatinib and ponatinib (Hegde et al., Leukemia 2022, PMID 34711926).

Estrogen Receptor alpha (ERa, gene name ESRI) is among the genes upregulated in leukemic cells resistant to IODVA1 treatment. Applicant thus hypothesized that IODVA1 will synergize with ERa degraders (such as, for example, fulvestrant) to treat high-risk leukemia. Applicant found that, using five different patient-derived xenograft (PDX) models of B-cell acute lymphoblastic leukemia (B-ALL), a combination of IODVA1 and fulvestrant, or sequential treatment with fulvestrant following IODVA1 treatment, is superior to the standard-of-care (ponatinib alone or in combination with IODVA1 or fulvestrant). Thus, disclosed are novel methods of treating cancers via administration of IODVA1 in combination with an ERa degrader. In embodiments, IODVA1 and an ERa degrader can be administered in combination to treat leukemia. In embodiments, IODVA1 and an ERa degrader can be administered in combination to treat a solid tumor. In embodiments, IODVA1 and an ERa degrader can be administered in combination to treat breast cancer. In embodiments, IODVA1 and an ERa degrader be administered in combination to treat a cancer that express VAV3 and ERa.

### IODVA1 Compounds

IODVA1 compounds and the manufacture of IODVA1 compounds are described in, for example WO2020227202A1, Nassar et al., entitled “Compositions and methods for treating cancer,” published 12 Nov. 2020, the contents of which are incorporated herein it their entirety.

In one aspect, the methods may employ the use of a composition that may comprise a compound having the following structure:

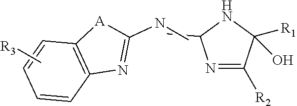

(referred to herein as “Compound 1,” or “an IODVA1 compound”) and a pharmaceutically acceptable carrier;
wherein A=NH, NR8, S, O, C═C, N═C, C═N
wherein R₁, R₂ are independently substituted or unsubstituted aryl or heteroaryl rings,
wherein R₃=singly or multiply substituted as H, D, Halo, CN, C1-C4 Alkyl, C1-C4 Alkoxy, C1-C4 Alkylsulfonyl, C1-C4 Alkyl amino, or C1-C4 mercapto, wherein R₈=H, M; and all tautomers thereof.

In one aspect, the compound may have the structure

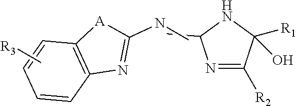

wherein A=NH, S; wherein R₁, R₂ are independently substituted or unsubstituted phenyl, pyridyl, furanyl, pyrimidinyl, triazinyl, or diazinyl rings; wherein R₃=singly or multiply substituted as H, D, Halo, CN, OH, OMe, OEt, SMe, SEt, SO₂Me, NHMe, NMe₂, Me, Et, or Pr;
and all tautomers thereof.

In one aspect, the compound may have the structure

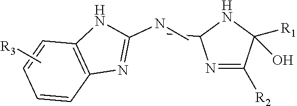

and wherein R₁, R₂ are independently substituted or unsubstituted phenyl, pyridyl, furanyl, pyrimidinyl rings; wherein R₃=singly or multiply substituted as H, D, Halo, CN, OH, OMe, OEt, SMe, SEt, SO₂Me, NHMe, NMe₂, Me, Et, or Pr, and all tautomers thereof.

In one aspect, the compound may have the structure

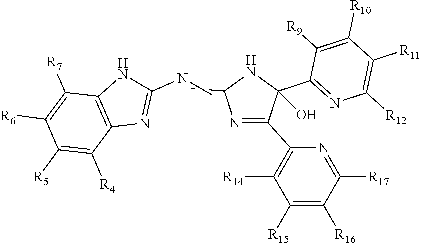

wherein R₄-R₁₇ are independently selected from H, D, Halo, CN, OH, OMe, OEt, SMe, SEt, SO₂Me, NHMe, NMe₂, Me, Et, or Pr; and all tautomers thereof.

In one aspect, the compound may have the structure

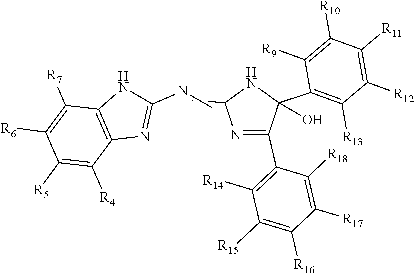

wherein R₄-R₁₇ are independently selected from H, D, Halo, CN, OH, OMe, OEt, SMe, SEt, SO₂Me, NHMe, NMe₂, Me, Et, or Pr; and all tautomers thereof.

In one aspect, the compound may have the structure

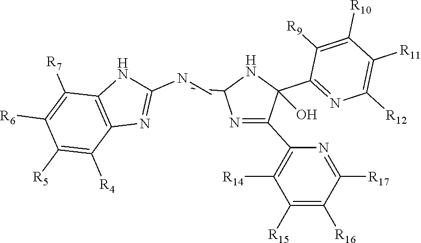

wherein R₄-R₇ are independently selected from H, D, F, CI, CN, OH, OMe, SMe, Me, or Et;
wherein R₉-R₁₇ are independently selected from H, D, F, CI, CN, OH, OMe, SO₂Me, NHMe, NMe₂, Me, or Et; and all tautomers thereof.

In one aspect, the compound may have the structure

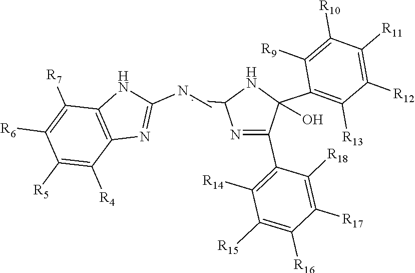

wherein R₄-R₇ are independently selected from H, D, F, CI, CN, OH, OMe, SMe, Me, or Et;
wherein R₉-R₁₇ are independently selected from H, D, F, CI, CN, OH, OMe, SO₂Me, NHMe, NMe₂, Me, or Et, and all tautomers thereof.

In one aspect, the compound may have the structure

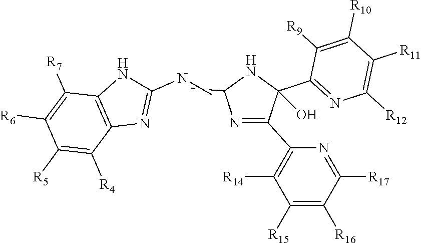

wherein R₄-R₇ are independently selected from H, D, F, OH, OMe, Me;
wherein R₉-R₁₇ are independently selected from H, D, F, CI, CN, OH, OMe, SO₂Me, NHMe, NMe₂, Me, or Et, and all tautomers thereof.

In one aspect, the compound may have the structure

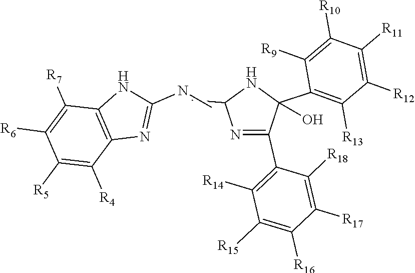

wherein R₅, R₆ are independently selected from H, D, F, CI, OH, OMe, or Me;
wherein R₉-R₁₇ are independently selected from H, D, F, CI, OH, OMe, or Me;
wherein each ring bears≤2 non-H substituents; and all tautomers thereof.

In one aspect, the compound may have the structure

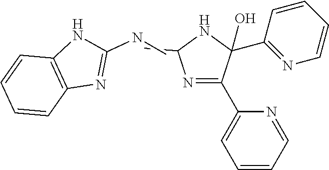

and all tautomers thereof.

In one aspect, the compound may have the structure

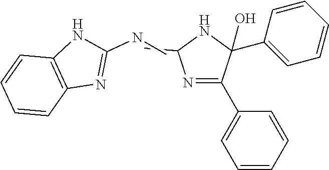

and all tautomers thereof.

In one aspect, the compound may have the structure and all tautomers thereof.

In one aspect, a composition comprising a compound having the structure

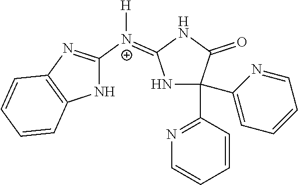 [SMILES: O=C1N/C(=[NH+]/c2nc3ccccc3[nH]2)NC1(c1ccccn1)c1ccccn1]

In one aspect, the compound may have the structure

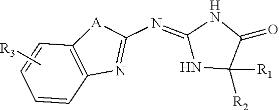

wherein A is selected from NH, NR8, S, O, C—C, N═C, C═N; wherein R₁, R₂ are independently substituted or unsubstituted* aryl or heteroaryl rings; wherein R₃ is singly or multiply substituted as H, D, Halo, CN, C1-C4 Alkyl, C1-C4 Alkoxy, C1-C4 Alkylsulfonyl, C1-C4 Alkyl amino, or C1-C4 mercapto; wherein R₈ is H or Me; and all tautomers thereof.

In one aspect, the compound may have the structure

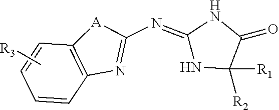

wherein A is NH or S; wherein R₁, R₂ are independently substituted or unsubstituted phenyl, pyridyl, furanyl, pyrimidinyl, triazinyl, or diazinyl rings; wherein R₃ is singly or multiply substituted as H, D, Halo, CN, OH, OMe, OEt, SMe, SEt, SO₂Me, NHMe, NMe₂, Me, Et, or Pr; and all tautomers thereof.

In one aspect, the compound may have the structure

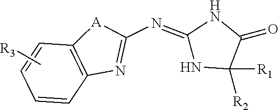

wherein R₁, R₂ are independently substituted or unsubstituted phenyl, pyridyl, furanyl, pyrimidinyl rings; wherein R₃ is singly or multiply substituted as H, D, Halo, CN, OH, OMe, OEt, SMe, SEt, SO₂Me, NHMe, NMe₂, Me, Et, or Pr; and all tautomers thereof.

In one aspect, the compound may have the structure

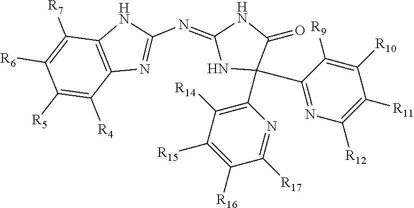

wherein R₄-R₁₇ are each independently selected from H, D, Halo, CN, OH, OMe, OEt, SMe, SEt, SO₂Me, NHMe, NMe₂, Me, Et, or Pr; and all tautomers thereof.

In one aspect, the compound may have the structure

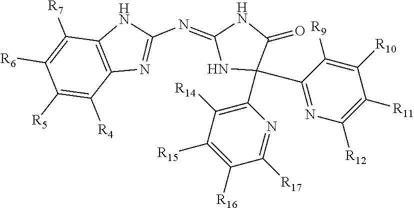

wherein R₄-R₁₇ are each independently selected from H, D, Halo, CN, OH, OMe, OEt, SMe, SEt, SO₂Me, NHMe, NMe₂, Me, Et, or P; and all tautomers thereof.

In one aspect, the compound may have the structure

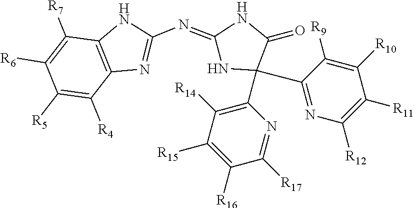

wherein R₄-R₇ are each independently selected from H, D, F, CI, CN, OH, OMe, SMe, Me, or Et;
wherein R₉-R₁₇ are each independently selected from H, D, F, CI, CN, OH, OMe, SO₂Me, NHMe, NMe₂, Me, or Et; and all tautomers thereof.

In one aspect, the compound may have the structure

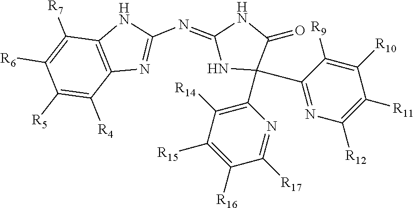

wherein R₄-R₇=H, D, F, CI, CN, OH, OMe, SMe, Me, or Et; wherein R₉-R₁₇=H, D, F, CI, CN, OH, OMe, SO₂Me, NHMe, NMe₂, Me, or Et, and all tautomers thereof.

In one aspect, the compound may have the structure

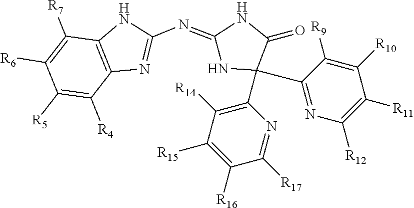

wherein R₄-R₇=Independently H, D, F, OH, OMe, Me; wherein R₉-R₁₇ may be independently selected from H, D, F, CI, CN, OH, OMe, SO₂Me, NHMe, NMe₂, Me, or Et, and all tautomers thereof.

In one aspect, the compound may have the structure

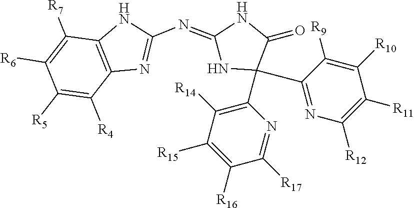

wherein R₄-R₇=Independently H, D, F, CI, OH, OMe, or Me; wherein R₉-R₁₂ and R₁₄-R₁₇ may be independently selected from H, D, F, CI, OH, OMe, or Me. Each ring bearing≤2 non-H substituents; and all tautomers thereof.

In one aspect, a composition comprising

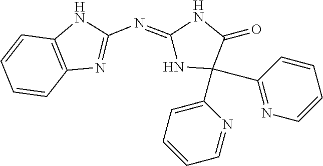 [SMILES: O=C1N/C(=N/c2nc3ccccc3[nH]2)NC1(c1ccccn1)c1ccccn1]

and all tautomers thereof, and a pharmaceutically acceptable carrier is disclosed.

In one aspect, a composition comprising

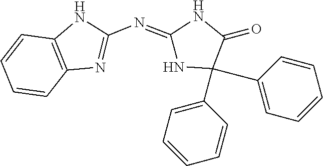 [SMILES: O=C1N/C(=N/c2nc3ccccc3[nH]2)NC1(c1ccccc1)c1ccccc1]

and all tautomers thereof, and a pharmaceutically acceptable carrier, is disclosed.

In embodiments, the disclosed methods include the administration of IODVA1 to an individual having, or suspected of having, a cancer.

### Selective ERα Degraders (SERDs)/Selective Estrogen Receptor Modulator (SERMs)

In embodiments, the disclosed methods include the administration of a selective ERα degrader (SERD). In embodiments, the SERD is fulvestrant.

In embodiments, the disclosed methods include the administration of a selective Estrogen Receptor Modulator (SERM). In embodiments, the SERM is tamoxifen. In embodiments, the SERM is raloxifene. In embodiments, the SERM is toremifene.

### Cancer Types

In embodiments, the disclosed methods are used to treat a cancer. In embodiments, the cancer is a “high-risk” cancer. In embodiments, the cancer is a cancer that is refractory to a first line cancer treatment. In embodiments, the cancer is a cancer that expresses VAV3 and ERa.

In embodiments, the cancer is a H/K/NRAS-driven cancer. In embodiments, the cancer is B-cell acute lymphoblastic leukemia (B-ALL). In embodiments, the cancer is leukemia. In embodiments, the cancer is lung cancer. In embodiments, the cancer is non-small cell lung cancer (NSCLC). In embodiments, the cancer is a solid tumor. In embodiments, the cancer is a triple-negative breast cancer (TNBC), hormone-resistant triple negative breast cancer. In embodiments, the cancer is treatment-resistant B-ALL.

In embodiments, the cancer is a VAV3-driven cancer.

In embodiments, the cancer is selected from blood cancer, pancreatic cancer, colon cancer, rectal cancer, colorectal cancer or lung cancer. In embodiments, the blood cancer is selected from acute myeloid leukemia or acute lymphocytic leukemia. In embodiments, the lung cancer is selected from non-small cell lung cancer or small cell lung cancer.

In embodiments, the cancer is characterized by expression of guanine nucleotide exchange factor VAV3, of its active phosphorylated form, and estrogen receptor alpha (ERa).

### Administration

In embodiments, sequential administration means that the residual tumor in the patient after the administration of the first active is minimum. In embodiments, the SERM and/or SERD is administered in a single dosage form with the IODVA1. In embodiments, the SERM and/or SERD is administered in a separate dosage form with the IODVA1.

In embodiments, the IODVA1 is administered at least one week prior to administration of a SERM and/or SERD. In embodiments, the IODVA1 is administered at least two weeks prior to administration of a SERM and/or SERD. In embodiments, the IODVA1 is administered at least three weeks prior to administration of a SERM and/or SERD. In embodiments, the IODVA1 is administered at least four weeks prior to administration of a SERM and/or SERD. In embodiments, the IODVA1 is administered more than four weeks prior to administration of a SERM and/or SERD. In other words, in embodiments, the SERM and/or SERD is administered at least one week following administration of a dose of IODVA1. In further embodiments, the SERM and/or SERD is administered at least two weeks following administration of a dose of IODVA1. In further embodiments, the SERM and/or SERD is administered at least three weeks following the administration of a dose of IODVA1. In further embodiments, the SERM and/or SERD is administered at least four weeks following the administration of a dose of IODVA1. In further embodiments, the SERM and/or SERD is administered at least five weeks following administration of a dose of IODVA1. In further embodiments, the SERM and/or SERD is administered at least six weeks following the administration of a dose of IODVA1. In further embodiments, the SERM and/or SERD is administered at least seven weeks following the administration of a dose of IODVA1. In further embodiments, the SERM and/or SERD is administered at least eight weeks following the administration of a dose of IODVA1. The SERM and/or SERD can be administered to the individual after a desired treatment result is observed in response to the IODVA1 treatment. The desired treatment result can take a variety of forms, which can be recognized by one of ordinary skill in the art. For example, in embodiments, the SERM and/or SERD is administered after, via a clinical assessment, the tumor and/or cancer that is being treated is observed to be maximally treated via the IODVA1. In embodiments, the SERM and/or SERD is administered after the residual tumor and/or cancer being treated with IODVA1 has decreased and has reached a minimum size. In embodiments, the SERM and/or SERD is administered after reaching a therapeutic plateau in response to the IODVA1. In embodiments, the SERM and/or SERD is administered after the tumor reaches minimal residual disease (MRD) in response to the IODVA1. In embodiments, the SERM and/or SERD is administered after tumor reduction cessation is observed in response to the IODVA1. In embodiments, the SERM and/or SERD is administered after the tumor stabilizes or enters a maintenance phase in response to the IODVA1. In embodiments, the SERM and/or SERD is administered after the tumor burden is minimized in response to the IODVA1. In embodiments, the SERM and/or SERD is administered after a defined minimal threshold of tumor reduction is reached in response to the IODVA1.

In embodiments, the SERM/SERD is Fulvestrant (sold under the brand name Faslodex™), and is administered, sequentially, after treatment with IODVA1. In embodiments, the cancer being treated is leukemia that relies on VAV3 for proliferation. In embodiments, the cancer being treated is a solid tumor that relies on VAV3 for proliferation

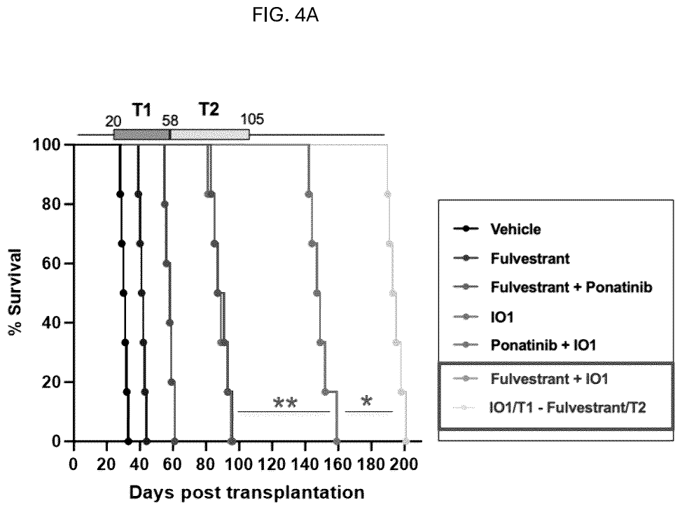

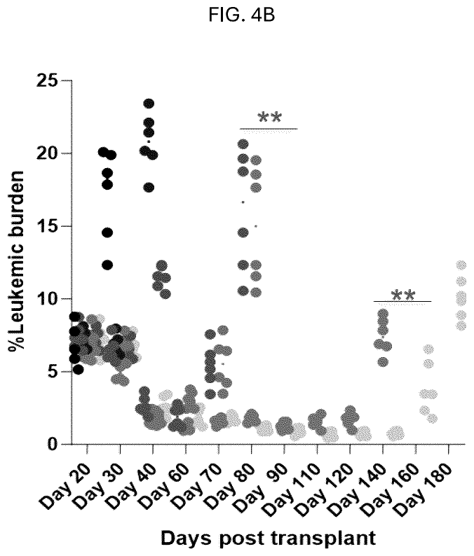

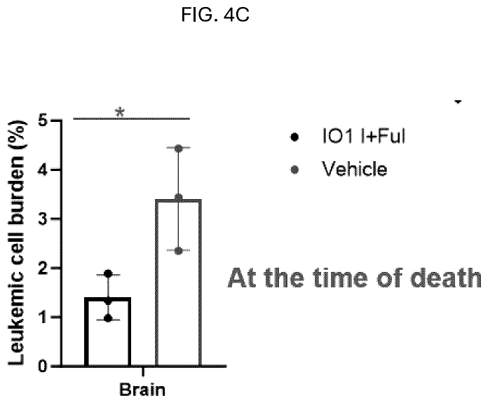

In one example, sequential IODVA1 treatment (days 20-51) followed by once weekly fulvestrant treatment (days 51-105) significantly prolongs the survival of both PDX models and decreases leukemic burden (FIGS. 4A, 6-8).

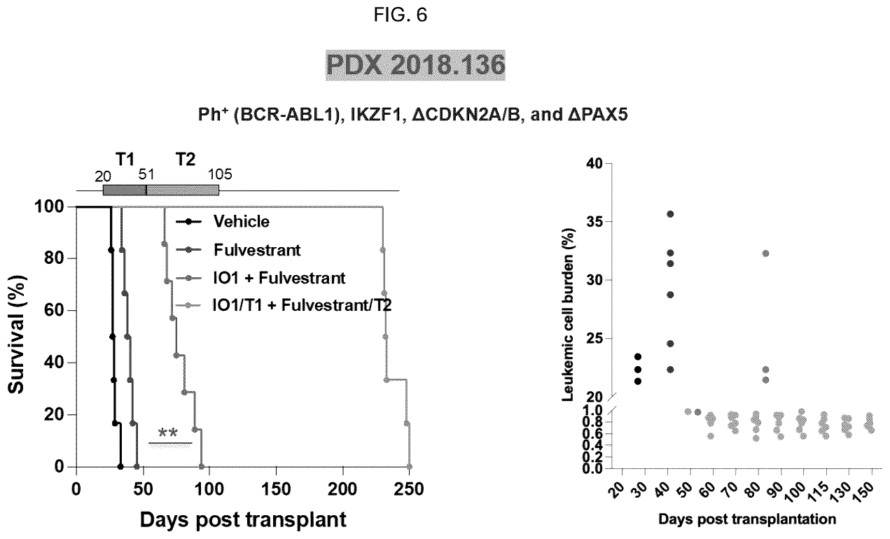

Leukemic mice survival is increased to day 250 even though treatment ceased by day 105 and oncogenic driver and genetic background are different (FIGS. 6 & 7). In both PDX models, the sequential treatment is superior to the IODVA1/fulvestrant concurrent combination. Moreover, in both PDX models, fulvestrant alone is not efficacious and the mice die of leukemia.

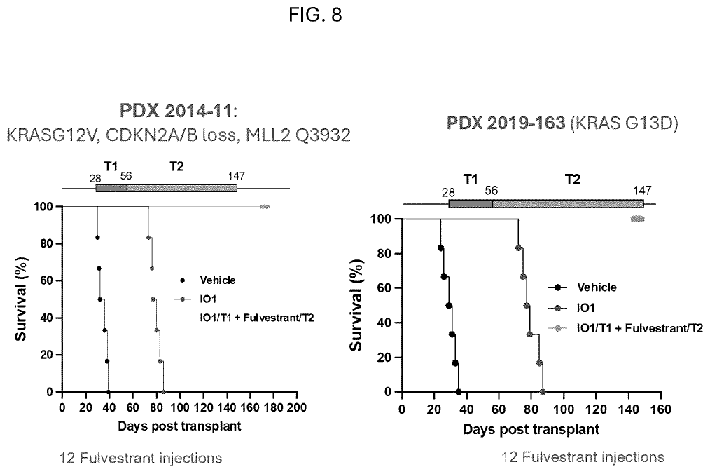

Additional testing involved IODVA1/fulvestrant to determine whether sequential treatment is efficacious in models of oncogenic KRAS-driven B-ALL (FIG. 8). Oncogenic mutations in KRAS are common in leukemia resistant to current therapies. KRAS is one of the most common mutated oncogenes in human cancer. Recently, small molecule inhibitors of KRAS have been developed but mechanisms of resistance to these mutants is also common.

Mouse models of PDX 2014-11 and 2019-163 with KRAS G12V and G13D mutations were generated. For each PDX model, mice were divided into 5 groups of 3 males and 3 females each and treated with vehicle control, IODVA1 alone between days 28 and 56 (T1 treatment), and the sequential combination of fluvestrant with both compounds (T1 and T2).

It is proposed that sequential treatment (i.e. IODVA1 first followed by fulvestrant) can be generalized to other types of leukemia and solid tumors that rely on VAV3 for proliferation.

### Simultaneous Administration

In embodiments, the administration of a VAV3-inhibitor such as IODVA1 and the administration of one or both of a SERM and a SERD are simultaneous. Simultaneous administration may be carried out by mixing the compounds prior to administration or by administering the compounds at the same point in time but at different anatomic sites or using different routes of administration. The phrases “concurrent administration,” “co-administration,” “simultaneous administration,” and “administered simultaneously” mean that the compounds are administered in combination. In embodiments, the treatment is carried out for at least two weeks, or at least three weeks, or at least four weeks, or at least five weeks, or at least six weeks, said administration being concurrently with or following IODVA1 administration.

### Dosage/Dosage Forms

The dosage regimen for the compounds and/or compositions containing the compounds is based on a variety of factors, including the type, age, weight, sex and medical condition of the patient; the severity of the condition; the route of administration; and the activity of the particular compound employed. Thus, the dosage regimen may vary widely. In embodiments, the IODVA1, SERM, and/or SERD is each administered at a dose of from about 1 mg/kg to about 200 mg/kg, or from about 3 mg/kg to about 150 mg/kg, or from about 5 mg/kg to about 100 mg/kg, or from about 10 mg/kg to about 75 mg/kg, or from about 15 mg/kg to about 50 mg/kg. One of ordinary skill in the art will recognize that determination of the appropriate dosage will depend, in part, on the age, weight, and sex of the individual.

### Delivery

Administration of the actives as described herein may be performed according to any of the accepted modes of administration available to those skilled in the art. Illustrative examples of suitable modes of administration include oral, nasal, pulmonary, parenteral, topical, intravenous, injected, transdermal, and rectal. Oral administration may involve swallowing, so that the compound enters the gastrointestinal tract, or buccal or sublingual administration may be employed, by which the compound enters the blood stream directly from the mouth. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques. In embodiments, the actives may also be administered topically to the skin or mucosa, that is, dermally or transdermally. In embodiments, the actives can also be administered intranasally or by inhalation. In embodiments, the actives can be administered rectally or vaginally. In embodiments, the actives can be administered directly to the eye or ear.

### Pharmaceutical Compositions

The disclosed actives may be administered as a pharmaceutical composition in any suitable pharmaceutical form. Suitable pharmaceutical forms include solid, semisolid, liquid, or lyophilized formulations, such as tablets, powders, capsules, suppositories, suspensions, liposomes, and aerosols. In embodiments, the compositions comprise the compound (or the stereoisomer thereof, a pharmaceutically acceptable salt thereof, or a pharmaceutically acceptable salt of the stereoisomer) as an active ingredient, and a pharmaceutically acceptable carrier and optionally other adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In embodiments, the compounds or a prodrug or a metabolite or pharmaceutically acceptable salts thereof, can be combined as the active ingredient in an admixture with a pharmaceutical carrier according to conventional pharmaceutical compounding techniques. The carrier may take a wide variety of forms depending on the form of preparation desired for administration, e.g. oral or parenteral (including intravenous). In embodiment, the pharmaceutical compositions can be presented as discrete units suitable for oral administration such as capsules, cachets or tablets each containing a predetermined amount of the active ingredient. In embodiments, the compositions can be presented as a powder, as granules, as a solution, as a suspension in an aqueous liquid, as a non-aqueous liquid, as an oil-in-water emulsion or as a water-in-oil liquid emulsion. In embodiments, the compound or a pharmaceutically acceptable salt thereof, may also be administered by controlled release means and/or delivery devices.

In embodiments, the pharmaceutical carrier/excipient can be, for example, a solid, liquid or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets.

In embodiments, the composition comprising the disclosed actives are suitable for parenteral administration may be prepared as solutions or suspensions of the active compounds in water. In embodiments, a surfactant is included such as, for example, hydroxypropyl cellulose. Dispersions can also be prepared in glycerol, liquid polyethylene glycols, and mixtures thereof in oils. In embodiments, a preservative is included to prevent the detrimental growth of microorganisms.

In embodiments, the composition comprising the disclosed active is suitable for injectable use. In embodiments, the composition suitable for injectable use includes a sterile aqueous solution or dispersion. In embodiments, the composition is in the form of a sterile powder for the extemporaneous preparation of such sterile injectable solutions or dispersions. In embodiments, the injectable form is sterile and effectively fluid for use with a syringe. In embodiments, the composition is stable under the conditions of manufacture and storage. In embodiments, the composition is preserved against possible contaminating action of microorganisms such as bacteria and fungi. Exemplary carriers include a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound or pharmaceutically acceptable salts thereof, may also be prepared in powder or liquid concentrate form.

### Definitions

Unless otherwise noted, terms are to be understood according to conventional usage by those of ordinary skill in the relevant art. In case of conflict, the present document, including definitions, will control. Exemplary methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the treatment of a cancer using the IODVA1 and a SERM and/or SERD.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some aspects, the terms refer to humans. In further aspects, the terms may refer to children.

The term “compound,” as used herein, is also intended to include any salts, solvates, or hydrates thereof.

The term “alkyl” includes straight, branched chain, or cyclic alkyl groups, such as, but not limited to, methyl, ethyl, propyl, butyl, trifluoromethyl, and tetradecyl.

The term “alkoxy” includes straight, branched chain, or cyclic alkoxy groups, such as, but not limited to, methoxy, ethoxy, propoxy, butoxy, 2-methoxyethoxy, sec-butoxy, hexyloxy, and 2-ethylhexyloxy, tetradecyloxy groups.

The term “aryl” encompasses monocyclic and polycyclic aryl groups which contain only carbons on the first ring. The term “monocyclic aryl” refers to phenyl (where the ring only contains carbons), and the term “polycyclic aryl” refers to napthyl and anthracenyl, to phenyl rings having at least a second ring fused thereto, and to napthyl rings having a third ring fused thereto. In the case of a polycyclic aryl consisting of a phenyl ring having a second or third ring fused thereto, or a napthyl ring having a third ring fused thereto, the additional rings may be aromatic or non-aromatic carbocyclic or heterocyclic rings, provided that in such cases the point of attachment will be to the carbocyclic aromatic ring. For example, a subset of this aryl group is a polycyclic aryl group wherein the second ring is a “heteroaryl” which contains carbon atoms and at least one heteroatom selected from the group consisting of O, N, and S (provided that O and S cannot be adjacent to each other in the same ring). Alternatively, a ring carbon atom of the second and/or third further rings may be replaced with a carbonyl [—C(═O) group] (e.g., when such rings are non-aromatic). “Substituted aryl” refers to an aryl group substituted by one or more substituents, preferably 1 to 4 substituents (more preferably 1 or 2), at any point of attachment of any ring, selected from alkyl, substituted alkyl, and the substituents recited above for substituted alkyl groups.

Accordingly, examples of aryl groups that are of interest in forming compounds of the invention include:
The terms “heterocycle”, “heterocyclic” and “heterocyclo” refer to fully saturated, partially unsaturated, or fully unsaturated, including aromatic (i.e., “heteroaryl”) cyclic groups (for example, 3 to 7 membered monocyclic, 7 to 11 membered bicyclic, or 10 to 16 membered tricyclic ring systems) which have at least one heteroatom in at least one carbon atom-containing ring. Thus, the term “heteroaryl” is a subset of heterocyclo groups. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized, provided sulfur and oxygen are not adjacent to each other in the ring. (The term “heteroarylium” refers to a heteroaryl group bearing a quaternary nitrogen atom and thus a positive charge.) Additionally, one or more (preferably one) carbon ring atoms of the heterocyclo ring may, as valence allows, be replaced with carbonyl group, i.e.,—C(═O)—. The heterocyclic group may be attached to the remainder of the molecule at any heteroatom or carbon atom of the ring or ring system.

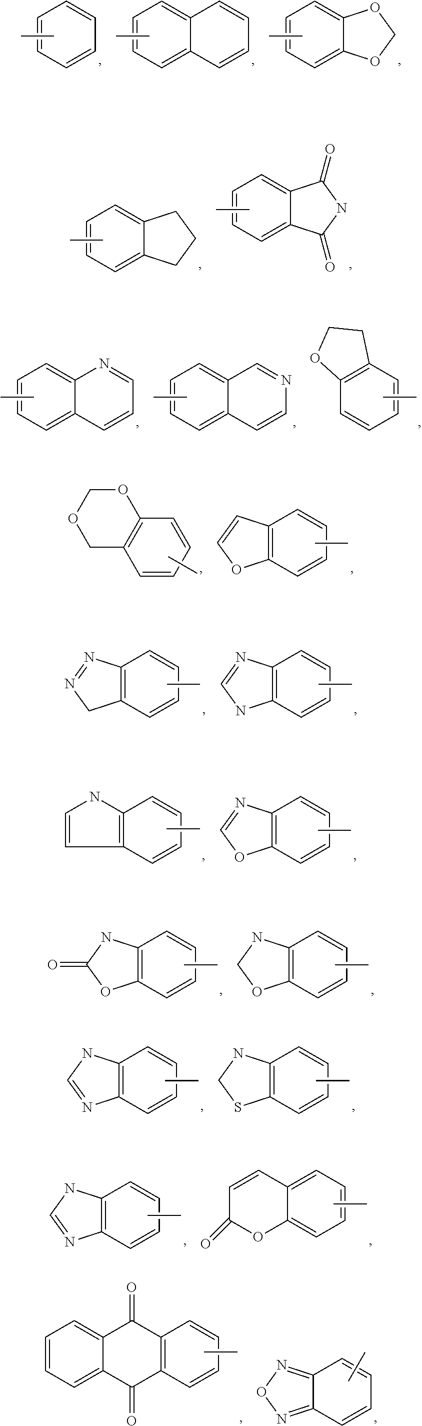

and, additionally, similar structures.

Exemplary monocyclic heterocyclic groups include those selected from the group consisting of ethylene oxide, azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like.

Exemplary bicyclic heterocyclic groups include those selected from the group consisting of indolyl, isoindolyl, benzothiazolyl, benzodioxolyl, benzoxazolyl, benzoxadiazolyl, benzothienyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo [2,3-c]pyridinyl, furo [3,2-b]pyridinyl] or furo [2,3-b]pyridinyl), dihydrobenzodioxinyl, dihydrodioxidobenzothiophenyl, dihydroisoindolyl, dihydroindolyl, dihydroquinolinyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, dibenzofuranyl, acridinyl, phenanthridinyl, xanthenyl and the like.

The term “heterocyclene” refers to bivalent heterocycle groups as defined above. “Substituted heterocycle”, “substituted heterocyclic” and “substituted heterocyclo” (such as “substituted heteroaryl”) refer to heterocycle, heterocyclic or heterocyclo groups substituted with one or more substituents, preferably 1 to 4 substituents, at any available point of attachment, wherein the substituents arc selected from those recited above for substituted cycloalkyl groups.

The term “group” is used, it is intended to encompass not only the substituent's unsubstituted form, but also its form further substituted with any substituent group or groups as herein mentioned, so long as the substituent does not destroy properties necessary for utility. Suitably, a substituent group may be halogen or may be bonded to the remainder of the molecule by an atom of carbon, nitrogen, oxygen, or sulfur.

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1

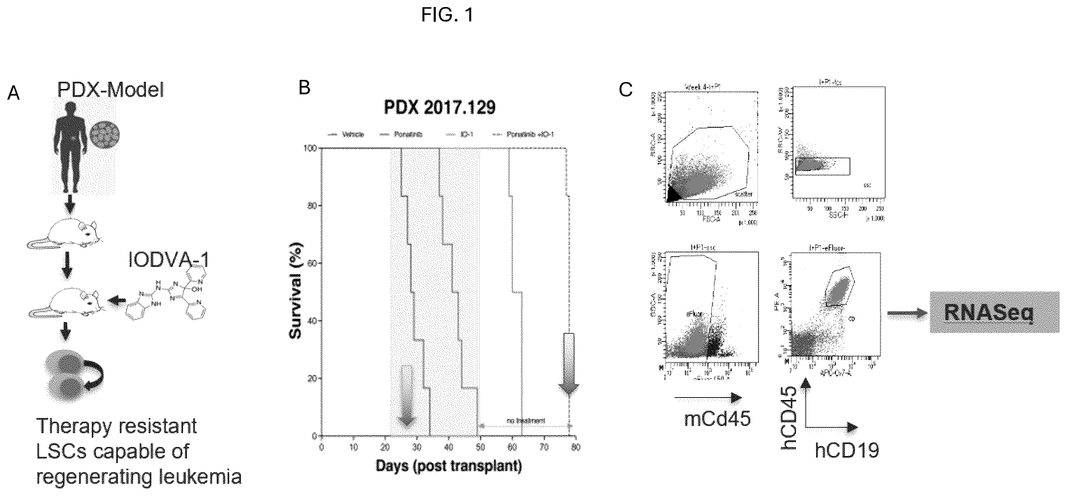

Applicant identified the most efficacious combination therapy using IODVA1, following the approach summarized in FIG. 1A-C. In brief, Applicant engrafted TKI-resistant Ph+ (BCR-ABL (T315I)) PDX model 2017-129 in NSG mice as previously described (FIG. 1A, Hegde et al., Leukemia 2022). When the leukemic burden reached ˜10%, mice were randomly divided into 4 groups of 6 mice each and treated with saline, IODVA1, ponatinib, and the IODVA1/ponatinib combination. Drugs were delivered in osmotic pumps (Alzet model 2002) that were placed subcutaneously. Mice were treated for 28 days (grey area in FIG. 1B) after which mice were observed in their cages. Human leukemic cells (mCD45-, hCD45+, hCD19+) were purified from bone marrow of treated mice by flow cytometry following one week of treatment and at relapse just before humanly sacking the mice (FIG. 1C). RNAs were generated from the following 7 groups: saline control, IODVA1, ponatinib, and combination at one week of treatment (IW, PW, and IPW) and IODVA1, ponatinib, and combination at relapse/end of treatment (IR, PR, and IPR).

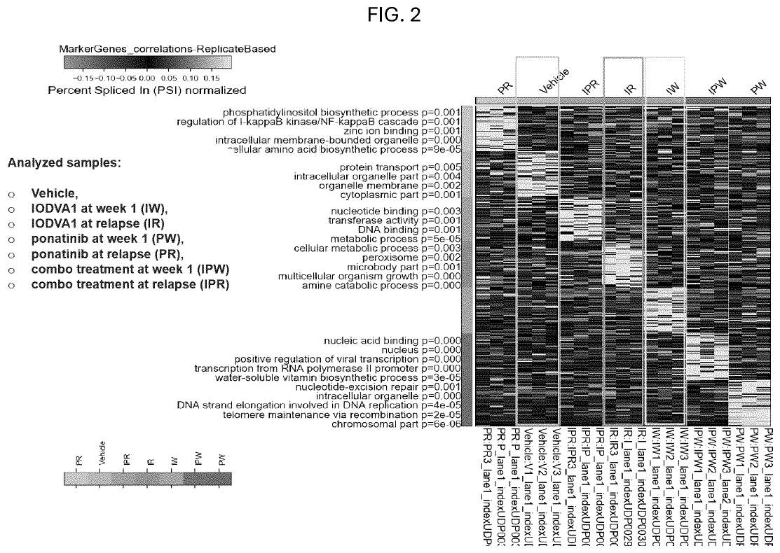

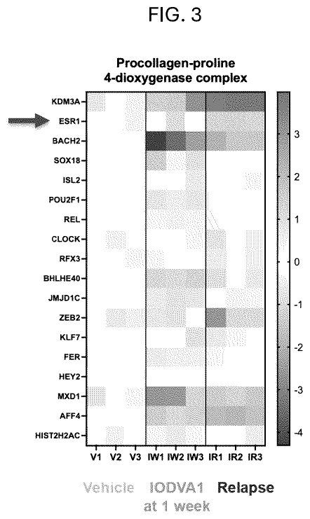

Transcriptomics analysis (FIG. 2) of the human leukemic cells revealed the following: Leukemic cells of mice treated with saline are characterized by activation of actin filament organization, cell-cell adhesion, and leukocyte migration which is consistent with IODVA1 targeting RAC activation; Leukemic cells of mice treated with IODVA1 for one week are characterized by histone modification signature; Leukemic cells of mice treated with IODVA1 at relapse are characterized among others by the upregulation of KDM3A and ERα (gene name ESR1, FIG. 3).

Inhibiting ERα post-IODVA1 treatment was an efficacious treatment for leukemia. The transcriptomics analysis of the human leukemic cells that relapsed following 4-weeks of IODVA1 treatment reveal that these cells rely among others on the estrogen receptor (ESR1/ERa) for proliferation (FIG. 3). This observation is in line with previous findings in the literature albeit in models of breast cancer showing that VAV3 and ERα interact (Lee et al., 2008, BMC Cancer, PMID 18518979) and that VAV3 mediates resistance to endocrine therapy (Aguilar et al., 2014, Breast Cancer Research, PMID 24886537). In addition, the histone demethylase KDM3A, which transcripts increase in relapsed leukemic cells (FIG. 3) was found to regulate ERα in breast cancer (Wade et al., Nucleic Acid Research 2015, PMID 25488809). Taken together, the data suggest that inhibiting ERα in combination or post IODVA1 treatment will decrease the survival the high-risk B-ALL.

Inhibiting ERα post-IODVA1 treatment significantly prolongs the survival of leukemic mice. To test the above hypothesis, Applicant generated engrafted PDX 2017-129 cells into irradiated NSG mice, waited for the leukemia burden to reach 10-15% and started treatment (FIG. 4A). Whereas all mice of treated groups die before or shortly after treatment ceased, mice in groups treated with the IODVA1/fulvestrant concurrently or sequentially survived post day 120 (FIG. 4A) demonstrating that the IODVA1/fulvestrant combination is a superior treatment for TKI-resistant B-ALL. The leukemic burden data from peripheral blood showed that the IODVA1/fulvestrant combination treatments decreased leukemic burden long after treatment ceased (FIG. 4B). The IODVA1/fulvestrant combination reduces leukemia invasion to the brain of treated mice (FIG. 4C). The effect of fulvestrant is specific for IODVA1 as the ponatinib/fulvestrant combo-treated mice (red line in FIG. 4A) succumb to the disease shortly post treatment stoppage. Fulvestrant alone is not efficacious as fulvestrant-treated mice (blue line in FIG. 4A) die during treatment, consistent with clinical practice in B-ALL that does not use ERa-SERM or SERD. Together, the data suggest that leukemic cells escaping IODVA1 treatment rely on ERα signaling for proliferation.

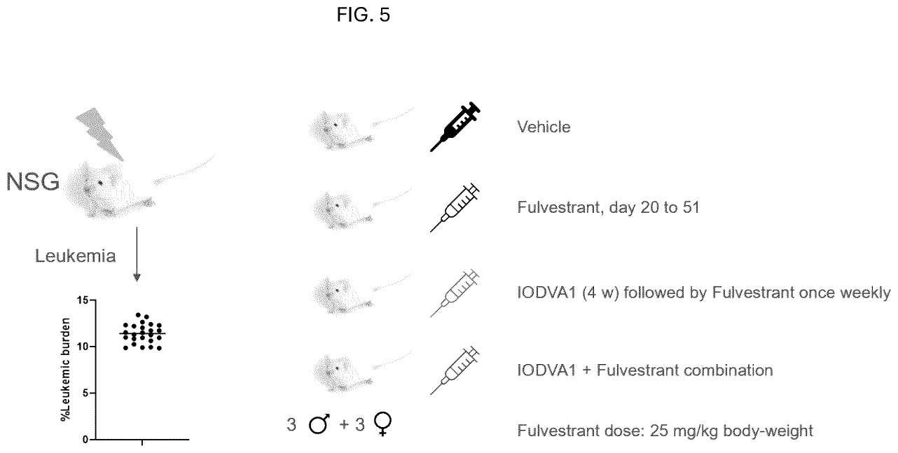

Inhibiting ERα post-IODVA1 treatment is oncogene driver independent. The success with IODVA1 and fulvestrant treatment with PDX 2017-129 (FIG. 4A & 4B) is not surprising as this treatment was suggested by the RNASeq data that were generated using this leukemia model. Applicant tested if this treatment applies to other B-ALL models with different driver oncogenes and genetic backgrounds. Xenograft mice models were generated with PDX B-ALL cells 2018-136 a Ph+BCR-ABL with IKZF1, ACDKN2A/B, APAX5 and 2016-79, a Ph-like characterized by the translocation IGH-CRLF2 and the JAK2 R₆₈₃G mutation (Table 1). These models were used as they are different from PDX 2017-129 and represent high-risk B-ALL with translocations commonly found in leukemia and that represent a challenge in the clinic. For each PDX model, mice were divided into 4 groups of 3 males and 3 females each and treated as shown in FIG. 5.

TABLE 1
PDX models used to test the IODVA1/fulvestrant treatment

| | | |
|---|---|---|
| PDX 2018-136 | Ph⁺ (BCR-ABL1) | IKZF1, |
| | | ΔCDKN2A/B, |
| | | ΔPAX5 |
| PDX 2016-79 | Ph-like | (IGH-CRLF2; |
| | | JAK2 R683G) |

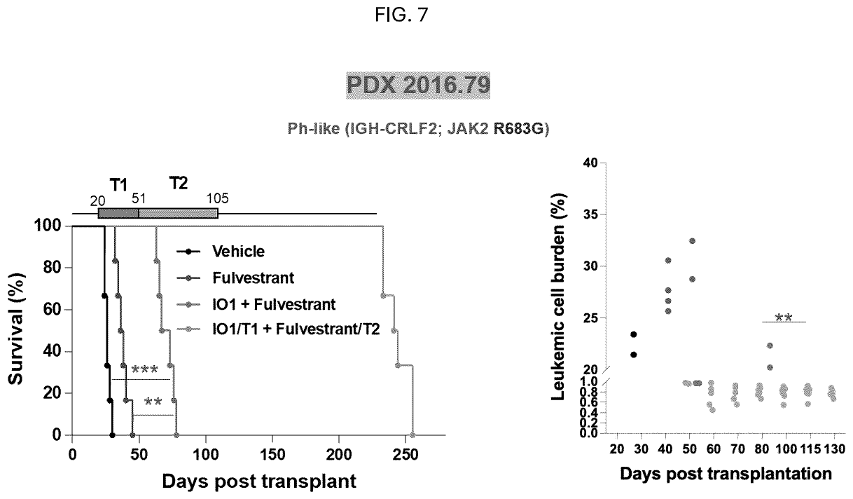

Inhibiting ERα post-IODVA1 is an efficacious treatment for B-ALL regardless of the driver oncogene. The sequential IODVA1 treatment (days 20-51) followed by once weekly fulvestrant treatment (days 51-105) significantly prolongs the survival of both PDX models and decreases leukemic burden (FIG. 6 & FIG. 7). Importantly, leukemic mice survival is increased to day 250 even though treatment ceased by day 105. In both PDX models, the sequential treatment is superior to the IODVA1/fulvestrant concurrent combination. In both PDX models, fulvestrant alone is not efficacious and the mice die of leukemia as enlarged spleens are noticed (data not shown).

Inhibiting ERα post-IODVA1 is an efficacious treatment for B-ALL regardless of the driver oncogene. Next, Applicant tested if the IODVA1/fulvestrant sequential treatment is efficacious in models of oncogenic KRAS-driven B-ALL. Oncogenic mutations in KRAS are common in leukemia resistant to current therapies. KRAS is one of the most common mutated oncogenes in human cancer. Recently, small molecule inhibitors of KRAS have been developed but mechanisms of resistance to these drugs is also common. Mouse models of PDX 2014-11 and 2019-163 with KRAS G12V and G13D mutations, respectively were generated. For each PDX model, mice were divided into 3 groups of 3 males and 3 females each and treated with vehicle control, IODVA1 alone between days 28 and 56 (T1 treatment), and the sequential combination of IODVA1 and fluvestrant (T1 and T2) as shown in FIG. 8. Although still ongoing, the data show that sequential treatment is efficacious in models of oncogenic KRAS.

Together, the data suggest that IODVA1 treatment pushes B-ALL cells regardless of their driver oncogene to rely on ERα for proliferation. In other words, leukemic cells become addicted to ERα signaling. Thus, including fulvestrant post IODVA1 treatment should further prolong survival of leukemic mice, reduce leukemic burden, and thus constitute a new and superior treatment for B-ALL. While in vivo data were generated with models of B-ALL, the sequential treatment (i.e. IODVA1 first followed by fulvestrant) is believed to be generalizable to other types of leukemia and solid tumors that rely on VAV3 for proliferation.

Mechanistic Studies of the VAV3/ERα functional interactions

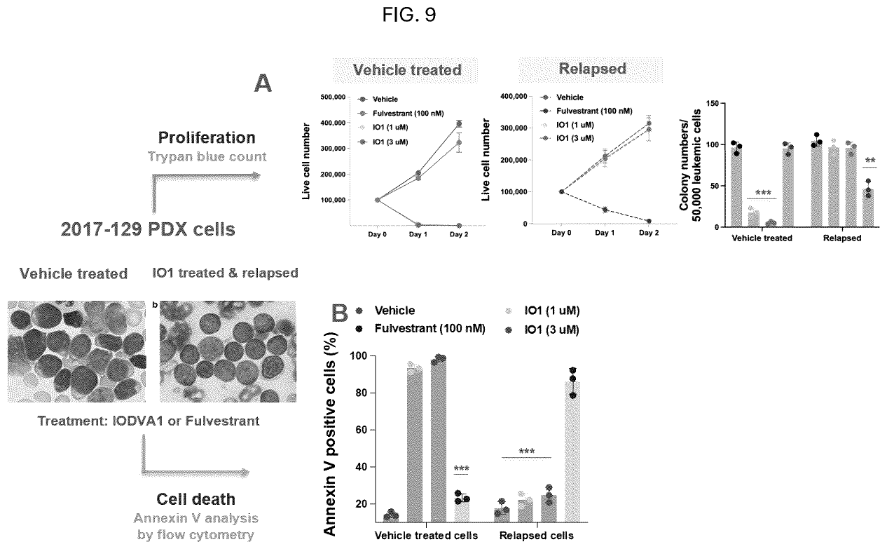

Biochemical studies of the functional interaction between VAV3 and ERa. To explain how IODVA1 treated leukemia becomes addicted to ERα inhibition, human leukemic cells were purified by flow cytometry from the spleen of PDX 2017-129 mice treated with vehicle control or with IODVA1 at the time of relapse (FIG. 4) and treated with IODVA1 (1 or 3 uM) or fulvestrant (1 nM). In proliferation assays, human leukemic cells from vehicle treated mice were sensitive to IODVA1 but did not respond to fulvestrant. Leukemic cells from IODVA1 treated mice on the other hand, were sensitive to fulvestrant but not to IODVA1 treatment (FIG. 9A). The decrease in proliferation is due to increase in cell death as shown by Annexin V staining (FIG. 9B). Taken together, the proliferation data show that leukemic cells switch their dependency to ERα for proliferation upon VAV3 inhibition.

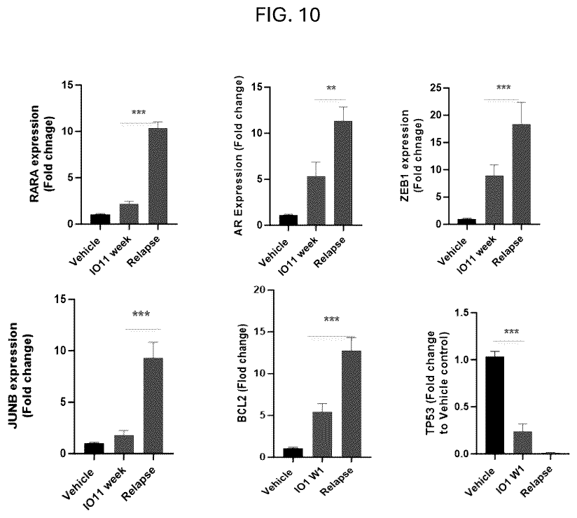

VAV3-inhibition increases the activity of ERa. The sensitivity of IODVA1-treated leukemic mice to fulvestrant suggests that the ERα is activated post VAV3-inhibition. The activity of ERα in the PDX 2017-129 cells obtained from mice treated with IODVA1 for one week or at relapse were controlled to vehicle control. Levels of downstream effector transcripts were quantified by QRT-PCR. Transcript levels of known ERα target genes such as the retinoic acid receptor alpha (RARA), androgen receptor (AR), zinc finger E-box binding homeobox 1 (ZEB1), JUNB, and BCL2 were significantly increased in leukemic cells from mice treated with IODVA1 and relapsed while transcripts of the tumor suppressor TP53 decreased (FIG. 10). Taken together, VAV3 inhibition by IODVA1 increases the activity of ERα as demonstrated by the increase in the transcript levels of its downstream targets.

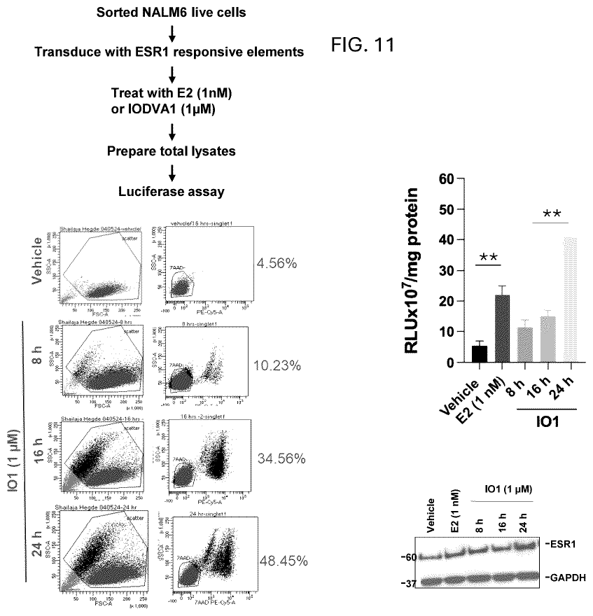

IODVA1 increases the transcriptional activity of ERα in model leukemic cells. In addition to the PCR data that showed increase in ERα activity following IODVA1 treatment of leukemic cells (FIG. 10), NALM6 cells were transduced with a plasmid expressing the estrogen response element (ERE, Walker P. et al., Nucleic Acids Res, 1984 PMID: 6504705) and the ERα transcriptional activity was quantified. Active ERα will bind to the ERE sequence, which is then read by a luciferase assay (FIG. 11). IODVA1 (1 uM) treatment of the NALM6-ERE cells for 24 h significantly increases the luciferase signal to levels similar or higher than estradiol/E2 (1 nM) treatment. Immunoblot analysis of the treated cells also show increase in the expression levels of the ERα protein. Together, the data show that IODVA1 stimulates the ERα transcriptional activity and protein levels, which in the long contributes to mechanisms of resistance.

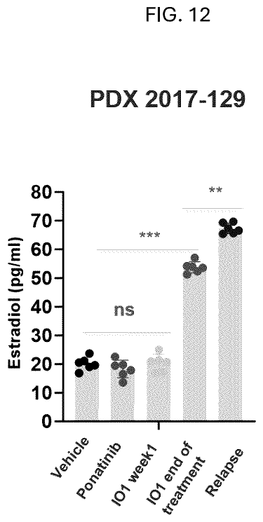

VAV3-pharmacological inhibition increases levels of the sex hormone estradiol/E2 in the plasma of treated mice. ERα is a nuclear receptor that is activated by the sex hormone 17ß-estradiol/E2. Given the sensitivity of the IODVA1-treated leukemic cells to fulvestrant and the increase in ERα activity in IODVA1-relapsed cells, the hypothesis that relapsed leukemic cells increase the levels of E2 to activate ERα was tested by measuring the levels of E2 in the plasma of PDX 2017-129 mice treated as in FIG. 4. Levels of estradiol/E2 were quantified in the plasma of the treated mice using an ELISA assay. Levels of E2 were significantly increased in the plasma of mice treated with IODVA1 at end of treatment and at relapse. E2 levels in plasma of mice treated with ponatinib or IODVA1 for one week were unchanged from levels of vehicle treated mice (FIG. 12).

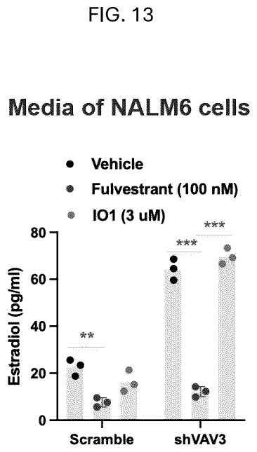

Genetical decrease of VAV3 levels increases levels of E2. Similarly, knocking down VAV3 (shVAV3) increases the levels of estradiol/E2 in the culture media of leukemic NALM6 cells treated with vehicle control or IODVA1 (FIG. 13). Interestingly, treating the shVAV3 NALM6 with fulvestrant decreases levels of E2 suggesting a feedback forward loop between ERα activity and E2 levels or that ERα regulates E2 levels. Taken together, VAV3 inhibition increases the activity of ERα by increasing the levels of circulating plasma E2. This increase is specific to IODVA1 as no increase was observed for ponatinib.

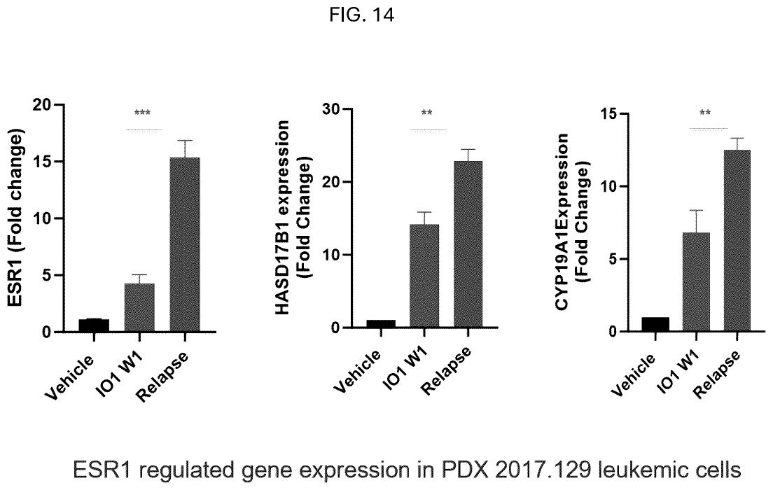

The increase in estradiol/E2 levels is due to increase in aromatase activity. The increase in levels of estradiol/E2 by leukemic cells suggests that the activity of the enzymes responsible for its synthesis, namely the 17ß-hydroxysteroid dehydrogenase 1 (gene name HSD17B1) and the estrogen synthase/aromatase (gene name CYP19A1) have also increased. Transcript levels of ESR1, HSD17B1, and CYP19A1 were measured by QRT-PCR in human leukemic cells purified from PDX 2017-129 mice treated with vehicle control and IODVA1 at one week time and at relapse (FIG. 14). mRNA levels of the 3 genes were significantly increased in leukemic cells from mice treated with IODVA1 and relapsed. Taken together, VAV3 inhibition increases the transcript levels of ERα and of the enzymes that synthesize estradiol/E2 leading to increased E2 in the leukemia surrounding and to an increase in ERα activity contributing to resistance.

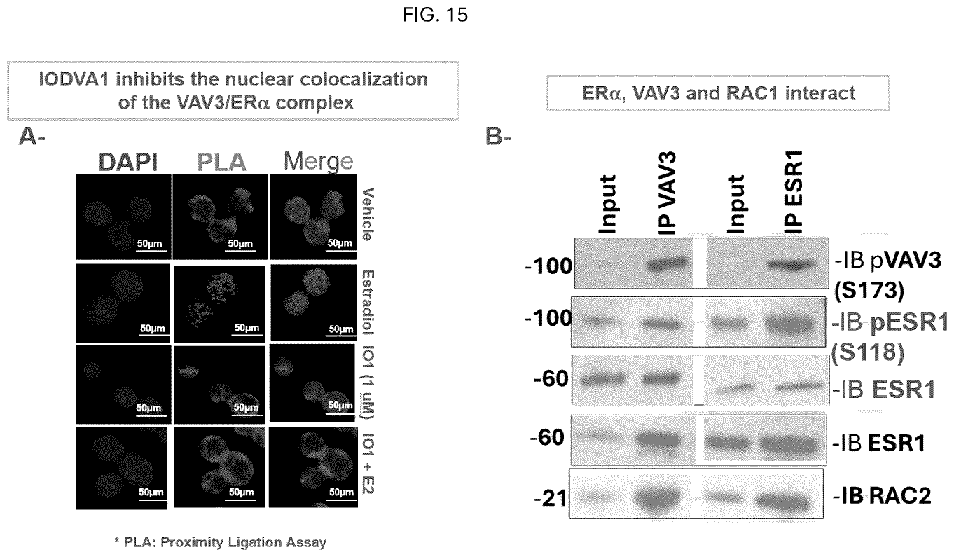

IODVA1 inhibits the nuclear co-localization of VAV3 and ERa. To test if VAV3 and ERα interact and the effect of IODVA1 on this interaction, the proximity ligation assay (PLA) and confocal microscopy was used to study the co-localization of these two proteins in NALM6 leukemic cells. As shown in FIG. 15A, a strong PLA (red) signal is localized to the cytoplasm when NALM6 cells are treated with vehicle control. This PLA signal shows that VAV3 and ERα interact. Following NALM6 cells treatment with estradiol/E2 (1 nM) for 45 min, the PLA signal localizes to the nucleus. When NALM6 cells are treated with IODVA1 (1 uM) for 1 hour followed by E2 for 45 min, the PLA signal is back to being mainly cytoplasmic. Together, the PLA data suggest that VAV3 and ERα interact in the cytoplasm. This protein complex moves to the nucleus following cell activation with E2. IODVA1 does not interfere with the complex formation but inhibits its translocation to the nucleus.

VAV3 and ERα interact. To further validate the VAV3/ERα interaction, the VAV3 and the ERα complexes were immunoprecipitated separately from NALM6 cells lysates. As shown in FIG. 15B, ERα is enriched in VAV3 precipitates and VAV3 is enriched in ERα precipitates confirming the interaction between these two proteins. Interestingly, the active phosphorylated form of both proteins (i.e. pY173VAV3 and pS118ERa) is enriched in both complexes. Immunoblotting for the small GTPase RAC2, the downstream target of VAV3 in the immunoprecipitated complexes confirmed its presence (FIG. 15B) suggesting that the VAV3/RAC/ERα are in a protein complex likely involving other proteins.

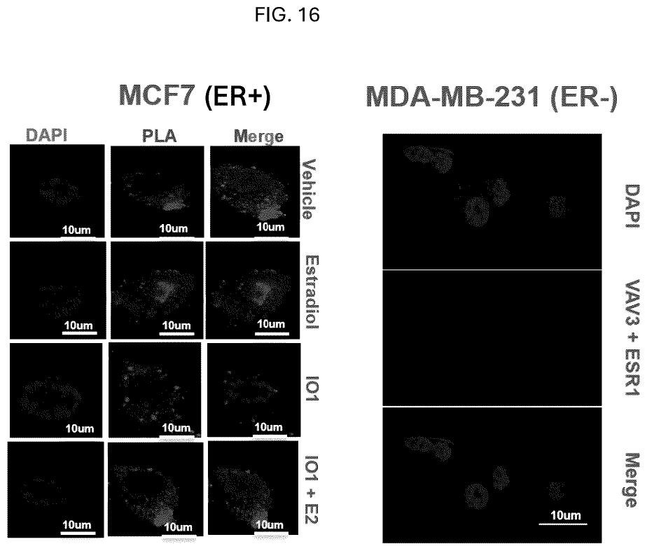

VAV3 and ERα interact in ER+breast cancer cells. To test if the interaction between VAV3 and ERα is specific to leukemic cells, the PLA assay was repeated by confocal microscopy in the ER+ and the ER-breast cancer cell lines MCF7 and MDA-MB-231, respectively. FIG. 16 shows that in MCF7 cells, the PLA signal is consistent with what was observed in the NALM6 cells. Briefly, the signal is cytoplasmic at steady state conditions but localizes to the nucleus following estradiol/E2 activation. Similar to what was observed in NALM6 cells, IODVA1 inhibits the localization of the PLA signal to the nucleus regardless of whether cells are treated first with E2 first or following incubation with IODVA1. The PLA signal is missing in the MDA-MB-231 cells (FIG. 16) as these cells lack ERa. Taken together, the data show that IODVA1 inhibits the localization of the VAV3/ERα complex into the nucleus in models of leukemia and breast cancers. It is posited that this is also true in other cancers.

In sum, Applicant has developed a novel combination therapy to treat high-risk B-cell acute lymphoblastic leukemia including leukemia resistant to current therapies. The novel therapy involves treating the leukemia first with a VAV3-inhibitor (e.g. IODVA1) followed by an estrogen receptor alpha (ERa) inhibitor or degrader such as fulvestrant. The therapy is not restricted to the use of fulvestrant but would also work with any other ERa-inhibitor/degrader such as tamoxifen. In embodiments, the therapy is independent of the driver oncogene but particularly effective with tumors that express VAV3, and to some extent ERa. This treatment is superior to FDA-approved and standard of care ponatinib used as monotherapy in relapsed/recurrent PDX models of leukemia. The disclosed therapy is expected to improve the outcome of cancer patients. The ability of the therapy to interfere with the localization of VAV3/ERα to the nucleus in cellular models of breast cancer especially hormone positive (ER+) breast cancer suggest that it will be efficacious in in vivo models of ER+breast cancer and other solid tumors expressing VAV3 and ERa. The data indicate that IODVA1 or a more potent analog in combination with an ERa-inhibitor should impact high-risk leukemia, ER+breast cancer, and any cancer expressing VAV3 and ERa, whether used alone or in combination with standard of care.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of treating a cancer comprising administering to said individual one or more compounds selected from 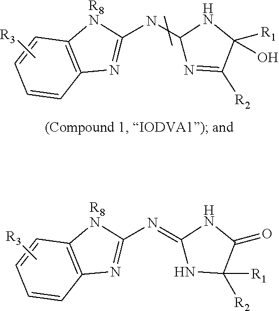
wherein R₁ and R₂ are independently substituted or unsubstituted aryl or heteroaryl rings;
wherein R₃ is singly or multiply substituted as H, D, Halo, CN, C1-C4 Alkyl, C1-C4 alkoxy, C1-C4 alkylsulfonyl, C1-C4 Alkyl amino, or C1-C4 mercapto; and
wherein R₈=H, Me; and all tautomers thereof;
and a selective estrogen receptor modulator (SERM) and/or selective estrogen receptor degrader (SERD), to an individual in need thereof.

2. The method of claim 1, wherein said compound is 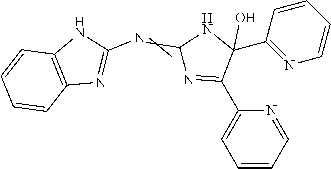
or a tautomer thereof.

3. The method of claim 1, wherein said compound is 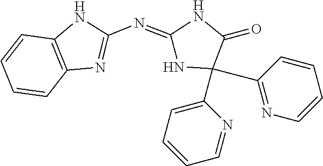 [SMILES: O=C1N/C(=N/c2nc3ccccc3[nH]2)NC1(c1ccccn1)c1ccccn1]
or a tautomer thereof.

4. The method of claim 1 the cancer being selected from an H/K/NRAS-driven cancer, B-cell acute lymphoblastic leukemia (B-ALL), leukemia, lung cancer, non-small cell lung cancer (NSCLC), solid tumor, breast cancer, triple-negative breast cancer (TNBC), hormone-resistant triple negative breast cancer, and combinations thereof.

5. The method of claim 1, the cancer being selected from de novo B-ALL and therapy resistant B-ALL.

6. The method of claim 4, the solid tumor being selected from lung cancer, breast cancer, subtypes thereof, and combinations thereof.

7. The method of claim 1, the cancer being TKI-resistant B-ALL.

8. The method of claim 1, the cancer being characterized by expression of guanine nucleotide exchange factor VAV3 (VAV3), of its activated phosphorylated form(s), and estrogen receptor alpha (ERa).

9. The method claim 1, wherein IODVA1 and the SERM and/or SERD are administered concurrently or sequentially.

10. The method of claim 1, the SERM and/or SERD being administered concurrently with IODVA1.

11. The method of claim 1, the SERM and/or SERD being administered following administration of IODVA1.

12. The method of claim 11, the SERM and/or SERD being administered at least one week following administration of IODVA1, or at least two weeks following administration of IODVA1, at least three weeks following administration of IODVA1, or at least four weeks following administration of IODVA1.

13. The method of claim 1, the SERD being selected from fulvestrant, elacestrant, giredestrant, imlunestrant, camizestrant, and clomiphene, and combinations thereof.

14. The method of claim 1, wherein said SERD is fulvestrant.

15. The method of claim 1, the SERM being selected from tamoxifen, raloxifene and toremifene, and combinations thereof.

16. The method of claim 1, the SERM and/or SERD being administered for at least two weeks, at least three weeks, at least four weeks, at least five weeks, or at least six weeks, the administration being concurrently with and/or following IODVA1 administration.

17. The method of claim 1, the administration being selected from oral, intravenous, subcutaneous, intramuscular, pulmonary (inhalation), topical, transdermal, intranasal, ophthalmic, buccal, epidural, intrathecal, and combinations thereof.

18. The method of claim 1, the IODVA1, SERM, and/or SERD each being administered at a dose of from about 1 mg/kg to about 200 mg/kg, or from about 3 mg/kg to about 150 mg/kg, or from about 5 mg/kg to about 100 mg/kg, or from about 10 mg/kg to about 75 mg/kg, or from about 15 mg/kg to about 50 mg/kg.

19. A pharmaceutical combination comprising a compound having the formula 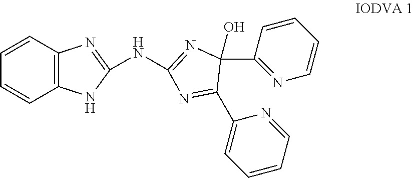 [SMILES: OC1(c2ccccn2)N=C(Nc2nc3ccccc3[nH]2)N=C1c1ccccn1]
and a selective estrogen receptor modulator (SERM) and/or selective estrogen receptor degrader (SERD).

20. The pharmaceutical combination of claim 19, wherein the IODVA1 compound and the SERM and/or SERD are provided in separate dosage forms.
